(19) United States
(12) Patent Application Publication
(10) Pub. No.: US 2004/0152190 A1
(43) Pub. Date: Aug. 5, 2004

(54) Method of separating and concentrating cells for kidney regfneration

(76) Inventor: Masaya Sumita, Shizuoka (JP)

Correspondence Address: YOUNG & THOMPSON, 745 SOUTH 23RD STREET 2ND FLOOR, ARLINGTON, VA, 22202

(21) Appl. No.: 10/479,236

(22) PCT Filed: May 31, 2002
(86) PCT No.: PCT/JP02/05348

(30) Foreign Application Priority Data
May 31, 2001 (JP) 2001-164362

(51) Int. Cl. A61K 45/00; C12N 5/08

(52) U.S. Cl. 435/369; 424/93.7

(57) ABSTRACT

A method and apparatus for separating and concentrating cells used for regenerating kidney tissues using a simple procedure in a short time is provided. The method comprises introducing a nucleated cells-containing fluid containing cells for kidney regeneration into a filter that can capture nucleated cells without capturing erythrocytes, and introducing a fluid for recovery into the filter, thereby recovering the cells for kidney regeneration captured by the filter. The concentrated cells for kidney regeneration are used for regenerating kidney tissues and treating kidney diseases.

### TECHNICAL FIELD

The present invention relates to a method and apparatus for separating and concentrating cells used for regenerating kidney tissues. The obtained cells can be used for treating deficiency of internal organs/tissues and various diseases and are useful in basic scientific fields such as immunology and cell biology.

### BACKGROUND ART

Although kidney transplantation is the only radical cure for chronic renal failure available at the present time, it is impossible to transplant kidneys to all chronic renal failure patients due to an insufficient number of donors. For this reason, patients with chronic renal failure must receive a blood purification treatment by artificial dialysis for survival. An increase in the number of patients receiving dialysis results in an increase in medical expenses and is a serious social problem. Although the burdens on patients have been reduced more than ever due to progress of the dialysis technology, the quality of life (QOL) of the patients receiving dialysis is unduly inferior to that of people who have received kidney transplantation.

In recent years, a regenerative medicine for curing diseases and/or defects of body tissues or internal organs (hereinafter referred to as “tissues”) by forming tissues in vivo or in vitro using cells that regenerate the tissues has been attracting great attention. Various studies have been undertaken in many countries (for example, The Tissue Culture Engineering (Gekkan Soshiki Baiyou Kougaku), Vol. 24, No. 4, Special Feature: Tissue Engineering I, April, 1998; Ibid, Vol. 24, No. 5, Special Feature: Tissue Engineering II, May, 1998). These studies have discovered that cells that regenerate the kidney are contained in the bone marrow (Journal of Clinical and Experimental Medicine (IGAKU NO AYUMI), Vol. 193, No. 1, April, 2000; The American Society of Nephrology, Abstract Collection A 1973, 2000, etc.). Non-target cells such as erythrocytes frequently coexist at the site in which these kidney-regenerating cells are present. It is therefore necessary to remove the non-target cells to collect the tissue-regenerating cells in a concentrated form. A technique such as density gradient centrifugation using Ficoll-Hypaque or similar or erythrocyte sedimentation using hydroxyethyl starch is usually employed to concentrate these cells. Both methods utilize centrifugation as the principle of separation and are commonly used at laboratory level in for example immunology, cell biology and laboratory medicine. However, these procedures are complicated. These methods are carried out on a clean bench. However, since the procedure requires a completely open system that can be sterilized only with difficulty, the methods are not by any means acceptable for clinical practices. For the regenerative medicine to proceed beyond an experimental medical treatment at a laboratory level and to develop into a routine medical practice, the separation-concentration procedure must be simplified and carried out in a closed, or only partially open, system.

In the field of the hematology, on the other hand, transplantation of hematopoietic stem cells to regenerate hematopoietic tissues (i.e. the bone marrow) has been established as a common medical practice. A filter method that is simple to handle has been proposed for concentration and separation of hematopoietic stem cells used in this field (for example, Japanese Patent Application Laid-open No. 8-104643).

### BRIEF DESCRIPTION OF THE DRAWINGS

FIG. 1 is a schematic illustration of a cell concentration apparatus for kidney regeneration.

FIG. 2 is a microphotograph showing engraftment of transplanted bone marrow cells in kidney tissue.

### DISCLOSURE OF THE INVENTION

An object of the present invention is to provide a method and apparatus for separating and concentrating cells used for regenerating kidney tissues using a simple procedure in a short time.

The present inventor has conducted extensive studies to achieve the above object. Based on the concept that achieving the above object of providing a simple, inexpensive, and time-saving procedure is very difficult if a conventional approach in the art of developing a separation technique using a surface antigen such as development of a novel monoclonal antibody is relied upon, the present inventor has undertaken a study on quite a novel technique without using a monoclonal antibody or similar. As a result, the present inventor has reached a surprising discovery that cells to regenerate kidney tissues can be separated and concentrated by using a filter for separation and concentration of hematopoietic stem cells. This finding has led to the completion of the present invention.

Therefore, the present invention relates to:

(1) a method for separating cells for kidney regeneration comprising introducing a nucleated cells-containing fluid containing cells for kidney regeneration into a filter that can capture nucleated cells without capturing erythrocytes, thereby causing the cells for kidney regeneration to be captured by the filter,

(2) a method for concentrating cells for kidney regeneration comprising introducing a nucleated cells-containing fluid containing cells for kidney regeneration into a filter that can capture nucleated cells without capturing erythrocytes, and then introducing a fluid for recovery into the filter, thereby recovering the cells for kidney regeneration captured by the filter,

(3) a method for regenerating kidney tissues comprising harvesting a cell suspension containing cells for kidney regeneration, passing the cell suspension through a filter that can capture nucleated cells without capturing erythrocytes, introducing a fluid for recovery into the filter to recover the cells for kidney regeneration captured by the filter, and using the recovered cells for regeneration of the kidney tissue,

(4) a method for treating kidney diseases comprising harvesting a cell suspension containing cells for kidney regeneration, passing the cell suspension through a filter that can capture nucleated cells without capturing erythrocytes, introducing a fluid for recovery into the filter to recover the cells for kidney regeneration captured by the filter, and administering the recovered cells for kidney regeneration to an individual suffering from a kidney disease,

(5) the method described in any one of (1) to (4) above, wherein the filter that can capture nucleated cells without capturing erythrocytes contains one or more formed products made from polyester, polyethylene, polypropylene, or polyurethane filled therein,

(6) the method described in above (5), wherein the formed product is a nonwoven fabric or porous sponge material,

(7) the method described in any one of (1) to (6) above, wherein the filter that can capture nucleated cells without capturing erythrocytes can pass platelets therethrough,

(8) an apparatus for separating cells for kidney regeneration comprising a cell separation filter filled with a cell capturing material that can capture nucleated cells without capturing erythrocytes in a container provided with at least an inlet port and an outlet port,

(9) an apparatus for concentrating cells for kidney regeneration comprising a cell separation filter filled with a cell capturing material that can capture nucleated cells without capturing erythrocytes and provided with at least an inlet port and an outlet port, a raw material cell suspension feeder connecting means connected at a point upstream of the inlet port of the cell separation filter, a fluid feeder connecting means for feeding a fluid into the cell separation filter at a point either upstream of the inlet port or downstream of the outlet port of the cell separation filter, and a cell recovering means connected with the fluid feeder connecting means via the cell separation filter on the opposite side of the fluid feeder connecting means at a point either upstream of the inlet port or downstream of the outlet port of the cell separation filter,

(10) the apparatus described in (8) or (9) above, wherein the cell separation filter contains one or more formed products made from polyester, polyethylene, polypropylene, or polyurethane filled therein,

(11) the apparatus described in (10) above, wherein the formed product is a nonwoven fabric or porous sponge material, and

(12) a fluid containing cells for kidney regeneration concentrated by the above method (2).

The present invention will be described in detail below.

Nucleated cells herein used refers to cells having a nucleus existing in tissues, internal organs, and body fluids (such as blood, lymph or similar) of animals (including humans) and includes, for example, leukocytes, granulocytes, neutrophils, eosinophilic leukocyte, basocytes, myelocytes, erythroblasts, lymphocytes, T-lymphocytes, B-lymphocytes, monocytes, hematopoietic stem cells, hemopoietic progenitor cells, and mesenchymal stem/progenitor cells. A nucleated cell-containing fluid containing cells for kidney regeneration used herein refers to a body fluid such as a bone marrow fluid, cord blood (including blood harvested not only from umbilical cord vessels but also from placenta vessels), peripheral blood, urine, or similar, a product obtained by subjecting the body fluid to certain treatment such as centrifugation, and a product obtained by suspending cells extracted from internal organs, such as kidney or various tissues such as muscle, in any fluid. The bone marrow fluid, for example, is a nucleated cell-containing fluid suitably used in the present invention.

The expression “without capturing erythrocytes” used herein means that erythrocytes pass through without being substantially captured, specifically, a phenomenon that 60% or more of the erythrocytes in the bone marrow pass through. The expression “can capture nucleated cells” used herein means that one half or more of the nucleated cells are captured, specifically, a phenomenon whereby 60% or more nucleated cells in the bone marrow are captured, but not necessarily all nucleated cells nor all types of nucleated cells are captured.

As a filter that can capture nucleated cells without capturing erythrocytes, a container having an inlet port and an outlet port filled with a nucleated cell capturing material that can substantially capture nucleated cells while allowing a substantial amount of erythrocytes to pass through, for example, can be mentioned.

Although any conventional cell capturing material can be used as the filter that can capture nucleated cells without capturing erythrocytes, materials preferably used in view of ease of fabrication, properties of being easily sterilized, and low cytotoxicity include, for example, synthetic polymers such as polyester, polyethylene, polypropylene, polystyrene, acrylic resin, nylon, polycarbonate, polyurethane, and similar, natural polymers such as cellulose, acetylcellulose, chitin derivative, chitosan, alginate, and similar, inorganic materials such as hydroxyapatite, glass, alumina, titania, and similar, and metals such as stainless steel, titanium, aluminum, and similar. Of these materials, polyester, polyethylene, polypropylene, and polyurethane are preferable due to easy availability in a medical grade and properties of being easily processed into capturing materials with a desired configuration.

These capturing materials may be used as is or may be used after optionally modifying the surface to increase selective permeability of cells. For example, to increase permeability of platelets, a method of coating a polymer with a nonionic hydrophilic group and a basic nitrogen-containing functional group proposed by WO 87/05812 can be given. To immobilize amino acids, peptides, saccharides, glycoproteins, and similar (including bio ligands such as antibodies, adhesion molecules, etc.), an immobilizing method using a haloacetamide as proposed by Japanese Patent Application Laid-open No. 02-261833, for example, can be suitably used.

Formed products such as particles, particle aggregates, fiber blocks, fabric, nonwoven fabric, porous sponge materials, and flat boards can be used as the capturing material configuration. Particles may be either porous particles or nonporous particles. Particle aggregates made from nonporous particles can be porous if particles aggregate with spaces among them. Fiber blocks, fabric, and nonwoven fabric can also be called porous materials because there are spaces between the fibers and threads thereof. The flat board refers to a plate made from a nonporous material. Porous materials such as porous particles, particle aggregates, fiber blocks, fabric, nonwoven fabric, and porous sponge materials are preferable due to their large surface area per unit volume. Among the porous materials, nonwoven fabric and porous sponge materials are preferable due to easy production and excellent flowability.

In the case of nonwoven fabric, the fiber diameter is usually 1.0-30 μm, preferably 1.0-20 μm, and more preferably 1.5-10 μm. If the fiber diameter is less than 1.0 μm, cells for kidney regeneration may be too firmly captured to obtain the concentrated fluid. If the fiber diameter is more than 30 μm, the possibility that the cells pass through the filter without being captured by the nonwoven fabric may increase. Either the smaller diameter or the larger diameter may unpreferably decrease the concentration rate.

In the case of porous sponge material, the pore diameter is usually 2.0-30 μm, preferably 2.5-25 μm, and more preferably 3.0-20 μm. If the pore diameter is less than 2.0 μm, flowability is impaired to an extent where it is sometimes difficult for a fluid to pass through the filter. If the pore diameter is more than 25 μm, the cell-capturing rate decreases, resulting in a low concentration rate.

As the material for the container to fill the nucleated cell-capturing material that does not capture erythrocytes therein, synthetic polymers such as polyethylene, polypropylene, polystyrene, acrylic resin, nylon, polyester, polycarbonate, polyacrylamide, polyurethane, and polyvinyl chloride, inorganic materials such as hydroxyapatite, glass, alumina, and titania, and metals such as stainless steel, titanium, and aluminum can preferably be mentioned in view of ease of fabrication, properties of being easily sterilized, and low cytotoxicity, for example. The vessel may have any shape such as a cuboid, cube, circular cylinder, oval cylinder, and similar. The inlet port may be provided at any position of the container allowing a fluid to be introduced into the uppermost layer of the filter. The outlet port may be provided at any position of the container allowing discharge of a fluid from the undermost layer of the filter.

The expression “cells for kidney regeneration” as used herein refers to cells capable of regenerating part or all the kidney tissues of animals (including humans), and includes, for example, kidney stem cells, kidney progenitor cells, mesenchymal stem and/or progenitor cells, vascular endothelial precursor cells, and kidney tubule precursor cells, but is not limited thereto.

In the present invention, a nucleated cell-containing fluid is first introduced into the filter, and then a fluid for recovery is introduced. Any fluid can be used as the fluid for recovery as long as the cells are not affected. Examples include a physiological saline solution, a buffer solution such as D-PBS (Dulbecco's phosphate buffer solution), HBSS (Hank's balanced salt solution) or similar, and a medium such as RPMI-1640, M199 or similar. Additives may optionally be added to the fluid for recovery with an objective of protecting cells, supplementing nutrients, providing coagulation resistance, preventing damage to cells in a frozen state, increasing viscosity (a high viscosity may be effective for increasing the recovery rate), and preventing infection. Examples of such additives include various blood serums such as FBS (fetal bovine serum), albumin, globulin, glucose, saccharose, trehalose, citric acid compounds, EDTA, dimethyl sulfoxide, dextran, polyvinyl pyrrolidone, glycerol, chitin derivatives, hydroxyethyl starch, gelatin and antibiotics. The expression “fluid for recovery” used herein includes not only a liquid, but also a mixture of a liquid and gas not adversely affecting cells such as air, argon, and nitrogen. The fluid for recovery may be introduced from either the same or the opposite direction of the flow of the nucleated cell containing fluid. The opposite direction is more preferable due to the tendency of giving a higher recovery rate.

In the present invention, the cell capturing material with cells captured therein may be used as is for transplantation or similar after dismantling the filter and taking out the cell capturing material without recovering the captured cells in the filter. For this purpose, the container preferably has a structure that can be easily opened for taking out the nucleated cell-capturing material therefrom by a simple procedure. If a filter container suitable for cell culture or preservation is used, the container can be used for culturing or preserving cells as is after introducing a medium and similar (for cell culture) or a freezing damage preventive and similar (for frozen cell preservation) without recovering the captured cells. In this instance, the filter container can function as a cell culture container or cell preservation container.

The method for regenerating the kidney of the present invention comprises causing a cell suspension containing cells for kidney regeneration to pass through the cell-separating filter and introducing the fluid for recovery into the filter to recover the cells for kidney regeneration captured by the filter. Before introducing the fluid for recovery, the filter may be rinsed to remove a small amount of erythrocytes and similar remaining in the filter. Any liquid can be used as a rinsing solution as long as the cells are not adversely affected. Examples include a physiological saline solution, a buffer solution such as Dulbecco's phosphate buffer solution (D-PBS) or Hank's solution (HBSS), and a medium such as RPMI1640 or M199. The rising solution may be introduced from either the same or opposite direction of the flow of the cell suspension. The same direction is more preferable due to the lower possibility of captured cells of leaking away.

In the method of regenerating the kidney of the present invention, although non-target cells such as erythrocytes pass through the cell-separating filter, such non-target cells may be recovered to use for another object. When the cell suspension is a bone marrow fluid obtained from a chronic renal failure patient, for example, the erythrocytes that are non-target cells permeated through the cell-separating filter may be recovered and preserved in a blood bag or similar, and may be used as an erythrocyte sample in basic scientific experiments or for collecting hemoglobin useful as a raw material for synthetic erythrocytes. Such erythrocytes can be transfused to patients as blood for transfusion. Such transfusion is preferably utilized for preventing anemia due to bone marrow harvest.

In the method for regenerating kidney tissues of the present invention, a cell suspension containing cells for kidney regeneration is harvested from an individual body. A suitable method is appropriately selected for harvesting cell suspension. For example, a method of using a bone marrow needle is employed for harvesting a bone marrow fluid, a method of using a centrifugal blood cell harvesting apparatus is employed for harvesting peripheral blood, and a method of using a syringe for harvesting blood is employed for harvesting cord blood.

The cells for kidney regeneration captured and recovered using the cell-separating filter of the present invention can be used not only for transplantation to either the same body from which the cells have been collected or another body, but also for in vitro regeneration of part or all of the kidney.

Examples of the in vitro regeneration of kidney tissues include, but are not limited to, a regeneration method comprising inoculating and culturing the cells in a “scaffold” of biodegradable or non-biodegradable material or a method for regenerating cells that can be regarded as mesangium cells reactive with angiotensin II via an AT1 receptor during culture with the addition of PDGF-B or retinoic acid.

The method for curing kidney diseases of the present invention comprises administering the cells for kidney regeneration obtained by the above-described method to an individual requiring kidney regeneration. The cells to be administered may be harvested from the same individual, a syngeneic but different individual, an individual of the same kind, or an individual of a different kind. When the cells are administered to an individual of the same or different kind of which the histocompatibility antigen is not identical with the individual from which the cells have been harvested, immune suppression treatment such as administration of an immunosuppressor is desirable. The kidney diseases in the present invention include glomerular nephritis, focal glomerular sclerosis, membranous nephropathy, membranoproliferative glomerulonephritis, IgA nephropathy, lupus nephritis, diabetic nephropathy, acute glomerulonephritis, minimal-change nephrotic syndrome, acute renal failure, chronic renal failure, and transplanted kidney disease, as well as damages and deficiency of the kidney due to accident, operation, and similar.

It is sufficient for the method of treating kidney diseases of the present invention that the kidney diseases are treated as a result of the method, irrespective of the treating mechanism. Specifically, not only the case in which the cells for kidney regeneration transplanted to an individual with a kidney disease differentiate in the site of transplantation to regenerate the kidney tissue, resulting in treating the kidney disease, but also an indirect case in which the transplanted cells for kidney regeneration do not differentiate in the site of transplantation, but act in some way on the cells or tissue existing in the site of transplantation resulting in treating the kidney disease is included.

The fluid containing cells for kidney regeneration of the present invention contains the cells for kidney regeneration concentrated using the above-described filter.

The cells for kidney regeneration obtained in the present invention can be used as is or after optional processing, such as purification, cultivation, activation, differentiation induction, amplification, gene introduction, and preservation in a frozen state, for curing various diseases and deficiencies, and for researches in basic scientific fields such as immunology and cell biology.

### BEST MODE FOR CARRYING OUT THE INVENTION

The present invention will be explained in more detail by Examples which are not intended to be limiting of the present invention.

### EXAMPLE 1

1. Cell-Separating Filter

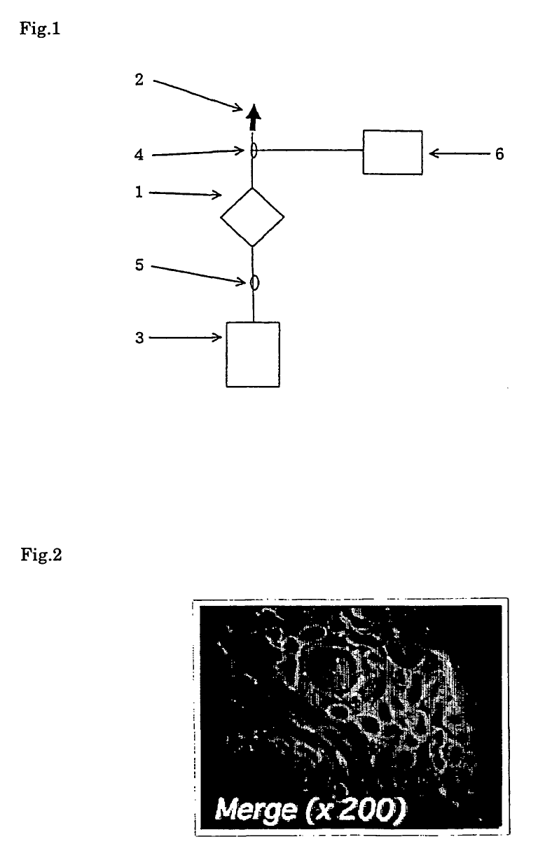

18 sheets of nonwoven polyester fabric (the density weight of the substrate per unit area (Metsuke): about 60 g/m2, bulk height: about 0.3 mm) made from fibers with an average fiber diameter of 2.3 μm and 16 sheets of nonwoven polyester fabric (Metsuke: about 100 g/m2, bulk height: about 0.47 mm) made from fibers with an average fiber diameter of 12 μm were stacked. The laminate was cut into 35 mm squares using a hand push cutter for use as a cell capturing material. A polycarbonate container having an external size (length×breadth×thickness) of 41 mm×41 mm×18 mm, with a diagonally opposite outlet port and inlet port, was packed with the cell capturing material so that the nonwoven polyester fabric with an average fiber diameter of 12 μm was on the outlet port side to obtain a cell-separating filter 1. A tube with a spike 2 on one end and a three-way cock 4 situated between the spike 2 and the filter 1 was connected to the inlet port side of the cell separation filter. Another tube was branched from the three-way cock 4 and connected to a cell recovering bag 6. Still another tube with a three-way cock 5 situated between the cell-separating filter 1 and an erythrocyte bag 3 was connected to the outlet port side of the cell separation filter 1, with the other end being connected to the erythrocyte bag 3, thereby fabricating a cell concentration apparatus shown in FIG. 1.

2. Bone-Marrow Cell Suspension

Bone marrow was harvested from the legs of four GFP (Green Fluorescent Protein) rats using a bone marrow harvesting solution (composition: M199/2% fetal bovine serum/gentamicin 2 μg/ml) and diluted with the same solution to obtain 60 ml of a bone marrow cell suspension. The solution was filled into a 200 ml blood bag.

3. Cell Concentration

The blood bag containing the bone marrow cell suspension obtained in procedure 2 (hereinafter referred to as “blood bag”) was connected to the spike 2 of the cell concentration apparatus fabricated in procedure 1. The three-way cock 4 was manipulated to connect the blood bag only with the cell-separating filter 1 and the three-way cock 5 was manipulated to connect the cell-separating filter 1 only with the erythrocyte bag 3 to filter the raw material cell suspension through the cell-separating filter by gravity and to recover filtered erythrocytes in the erythrocyte bag. Next, a 30 ml syringe (luer lock mouth) containing 25 ml of the bone marrow harvesting solution used in procedure 2 was connected to the three-way stopcock 5. The three-way cock 5 was manipulated to connect the syringe only with the cell-separating filter 1 and the three-way cock 4 was manipulated to connect the cell-separating filter 1 only with the cell recovering bag 6. Subsequently, the syringe plunger was pushed by hand to recover cells captured by the cell-separating filter 1 in to the cell recovering bag 6. The total period of time required for the procedure was 10 minutes.

4. Cell Transplantation

2.5 ml of a cell suspension obtained by concentrating the cells recovered in procedure 3 by conventional centrifugation was intravenously injected into the tails of four rats (not GFP rats).

5. Results

(1) Cell Separation

Non-target cells (mostly comprising erythrocytes, thus counted as erythrocytes) and nucleated cells recovered in the cell recovering bag were respectively counted using an automatic blood cell counter and a macroscopic counting method using Tuerk solution. Since the majority of non-target cells were erythrocytes, the number of erythrocytes was regarded as the number of non-target cells. The resulting non-target cell removal rate and nucleated cell recovery rate are shown in Table 1. Not all of the nucleated cells are useful for kidney regeneration, but the cells for kidney regeneration are included in the nucleated cells.

TABLE 1

| Erythrocytes | Raw material cell suspension | 5.5 × 10⁹ |
|---|---|---|
| | Cell recovering bag | 4.5 × 10⁸ |
| | Removal rate | 92% |
| Nucleated cells | Raw material cell suspension | 9.0 × 10⁸ |
| | Cell recovering bag | 6.2 × 10⁸ |
| | Removal rate | 69% |

(2) Transplantation

The rats were sacrificed 28 days after the transplantation and dissected to inspect the transplanted cells (that emit green fluorescence due to the GFP rat origin) in the kidney. As a result, presence of the transplanted cells was confirmed in the kidney interstitium and the glomerule. The experiment thus showed that the cells obtained from bone marrow by the concentration-separation technique of the present invention can regenerate the kidney. A microphotograph of the kidney tissue of the rat which was sacrificed and dissected is shown in FIG. 2, wherein white spots are transplanted bone marrow cells engrafted in the kidney tissue.

### INDUSTRIAL APPLICABILITY

As described above, since the method of the present invention can concentrate cells for kidney regeneration using a simple procedure in a short time, the method greatly contributes to development of basic scientific fields such as immunology and cell biology, and to assisting regenerative medicine to proceed past an experimental medical procedure at a laboratory level and to develop into a routine medical practice.

1. A method for separating cells for kidney regeneration comprising introducing a nucleated cells-containing fluid containing cells for kidney regeneration into a filter that can capture nucleated cells without capturing erythrocytes, thereby causing the cells for kidney regeneration to be captured by the filter.

2. A method for concentrating cells for kidney regeneration comprising introducing a nucleated cells-containing fluid containing cells for kidney regeneration into a filter that can capture nucleated cells without capturing erythrocytes, and then introducing a fluid for recovery into the filter, thereby recovering the cells for kidney regeneration captured by the filter.

3. A method for regenerating kidney tissues comprising harvesting a cell suspension containing cells for kidney regeneration, passing the cell suspension through a filter that can capture nucleated cells without capturing erythrocytes, introducing a fluid for recovery into the filter to recover the cells for kidney regeneration captured by the filter, and using the recovered cells for regeneration of the kidney tissues.

4. A method for treating kidney diseases comprising harvesting a cell suspension containing cells for kidney regeneration, passing the cell suspension through a filter that can capture nucleated cells without capturing erythrocytes, introducing a fluid for recovery into the filter to recover the cells for kidney regeneration captured by the filter, and administering the recovered cells for kidney regeneration to an individual suffering from a kidney disease.

5. The method according to any one of claims 1 to 4, wherein the filter that can capture nucleated cells without capturing erythrocytes contains one or more formed products made from polyester, polyethylene, polypropylene, or polyurethane filled therein.

6. The method according to claim 5, wherein the formed product is a nonwoven fabric or porous sponge material.

7. The method according to any one of claims 1 to 6, wherein the filter that can capture nucleated cells without capturing erythrocytes can pass platelets therethrough.

8. An apparatus for separating cells for kidney regeneration comprising a cell separation filter filled with a cell capturing material that can capture nucleated cells without capturing erythrocytes in a container provided with at least an inlet port and an outlet port.

9. An apparatus for concentrating cells for kidney regeneration comprising a cell separation filter filled with a cell capturing material that can capture nucleated cells without capturing erythrocytes and provided with at least an inlet port and an outlet port, a raw material cell suspension feeder connecting means connected at a point upstream of the inlet port of the cell separation filter, a fluid feeder connecting means for feeding a fluid into the cell separation filter at a point either upstream of the inlet port or downstream of the outlet port of the cell separation filter, and a cell recovering means connected with the fluid feeder connecting means via the cell separation filter on the opposite side of the fluid feeder connecting means at a point either upstream of the inlet port or downstream of the outlet port of the cell separation filter.

10. The apparatus according to claim 8 or 9, wherein the cell separation filter contains one or more formed products made from polyester, polyethylene, polypropylene, or polyurethane filled therein.

11. The apparatus according to claim 10, wherein the formed product is a nonwoven fabric or porous sponge material.

12. A fluid containing cells for kidney regeneration concentrated by the method according to claim 2.
